(19) United States
(12) Patent Application Publication
(10) Pub. No.: US 2024/0412105 A1
(43) Pub. Date: Dec. 12, 2024

(54) METHOD OF PREDICTING LITHIUM ION CONDUCTIVITY OF SOLID ELECTROLYTE

(71) Applicants: Hyundai Motor Company, Seoul (KR); Kia Corporation, Seoul (KR); Research & Business Foundation Sungkyunkwan University, Suwon (KR)

(72) Inventors: Sun Ho Choi, Incheon (KR); Yong Jun Jang, Seongnam, Gyeonggi-do (KR); Yong Gu Kim, Suwon, Gyeonggi-do (KR); Sang Heon Lee, Yongin, Gyeonggi-do (KR); Seong Hyeon Choi, Seongnam, Gyeonggi-do (KR); Sa Heum Kim, Seoul (KR); Sung Man Cho, Gwacheon, Gyeonggi-do (KR); Sang Uck Lee, Bucheon, Gyeonggi-do (KR); Ji Hoon Kim, Ansan, Gyeonggi-do (KR); Ji Won Lee, Suwon, Gyeonggi-do (KR); Ji Seon Kim, Suwon, Gyeonggi-do (KR)

(21) Appl. No.: 18/531,173

(22) Filed: Dec. 6, 2023

(30) Foreign Application Priority Data
Jun. 9, 2023 (KR) 10-2023-0074343

(51) Int. Cl. G06N 20/00

(52) U.S. Cl. CPC G06N 20/00

(57) ABSTRACT

Disclosed is a method of rapidly and precisely predicting lithium ion conductivity of a solid electrolyte. The method may include simulating a crystal structure of the solid electrolyte and creating training sets based on the crystal structure for machine learning; calculating a potential specific to the simulated crystal structure by machine learning using the training sets; and predicting the lithium ion conductivity of the solid electrolyte from the potential using molecular dynamics simulations.

### CROSS-REFERENCE TO RELATED APPLICATION

This application claims under 35 U.S.C. § 119 (a) the benefit of Korean Patent Application No. 10-2023-0074343 filed on Jun. 9, 2023, the entire contents of which are incorporated herein by reference.

### BACKGROUND

### (a) Technical Field

The present disclosure relates to a method of rapidly and precisely predicting lithium ion conductivity of a solid electrolyte.

### (b) Background

Secondary batteries are widely used in a lot of equipment from large-sized equipment, such as a vehicle, a power storage system, and the like, to small-sized equipment, such as a mobile phone, a camcorder, a notebook, and the like, in these days.

As the area of application of secondary batteries is broadened, demand for improvement of safety and high performance of the secondary batteries is increasing. A lithium secondary battery which is one kind of secondary batteries has advantages, such as a high energy density and a high capacity per unit area, compared to a nickel-manganese battery or a nickel-cadmium battery.

However, most of electrolytes used in the conventional lithium secondary batteries are liquid electrolytes, such as organic solvents. Therefore, safety issues, such as leakage of an electrolyte, risk of fire caused thereby, and the like, are constantly being raised.

Therefore, in order to increase safety of secondary batteries, interest in all-solid-state batteries using a solid electrolyte other than a liquid electrolyte is increasing. An all-solid-state battery, which uses a solid electrolyte instead of a liquid electrode, is advantageous in terms of safety because all components of the all-solid-state battery, including electrodes and electrolytes, are solids. Further, the all-solid-state battery may use lithium metal or a lithium alloy as an anode material, and thus it is known that it is advantageous in terms of performance of the battery, i.e., has a high energy density, high output, and a long lifespan, and therefore, research on all-solid-state batteries is ongoing, particularly, research on solid electrolytes having an argyrodite-type crystal structure is actively underway.

Research on synthesis of solid electrolytes having various crystal structures and compositions in order to develop solid electrolytes having high lithium ion conductivity requires a lot of time and effort. In order to overcome such a drawback there are many attempts to predict lithium ion conductivities of solid electrolytes through calculation.

Recently, most research on calculation of lithium ion conductivities of solid electrolytes uses the ab initio molecular dynamics (AIMD) method using density functional theory (DFT). However, the AIMD method is disadvantageous in that there is a big difference between an experimental value and a predicted value, a predicted crystal structure of a solid electrolyte is small, lithium ion conductivity is calculated at an assumed high temperature other than room temperature, and computational cost is high.

As another method, there is the molecular dynamics simulations. This method may solve most of the above-described problems, but is disadvantageous in that a calculation cannot be made unless there is a potential corresponding a structure and composition to be calculated.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present disclosure has been made in an effort to solve the above-described problems associated with the prior technology, and it is an object of the present disclosure to provide a method of predicting lithium ion conductivity of a solid electrolyte which solves drawbacks of density functional theory (DFT) and the molecular dynamics simulations using machine learning.

It is another object of the present disclosure to provide a method of rapidly and precisely calculating lithium ion conductivity of a solid electrolyte.

In one exemplary embodiment, a method of predicting lithium ion conductivity of a solid electrolyte. The method may include: simulating a crystal structure of the solid electrolyte and creating training sets based on the crystal structure for machine learning; calculating a potential specific to the simulated crystal structure by machine learning using the training sets; and predicting the lithium ion conductivity of the solid electrolyte from the potential using molecular dynamics simulations.

The training sets may be created using ab initio molecular dynamics (AIMD) method.

The potential specific to the simulated crystal structure may be a Moment Tensor Potential (MTP). The MTP may be used to calculate lithium ion diffusivity, which is then used to predict the lithium ion conductivity.

In one exemplary embodiment, a method of predicting lithium ion conductivity of a solid electrolyte having an argyrodite-type crystal structure and expressed as Li6-aPS5-aX1+a (0≤a≤1, and X=Cl, Br, or I) is provided. The method may include: simulating a plurality of compounds having different ratios of an element X occupying 4c sites comprised in the argyrodite-type crystal structure; assuming a virtual space comprising n×m×p cells (each of n, m and p being an integer in a range of 1 to 3), and simulating a random structure by differentially distributing the plurality of compounds to the cells based on thermodynamic stabilities of the plurality of compounds; creating training sets based on the random structure for machine learning; calculating a potential specific to the random structure by machine learning using the training sets; and predicting the lithium ion conductivity of the solid electrolyte from the potential using molecular dynamics simulations.

The training sets may be created using ab initio molecular dynamics (AIMD) method.

The compounds may comprise: a first compound having a ratio of the element X of 0% and a space group F4 3m; a second compound having a ratio of the element X of 25% and a space group R3m; a third compound having a ratio of the element X of 50% and a space group P2122; a fourth compound having a ratio of the element X of 50% and a space group P2 mm; a fifth compound having a ratio of the element X of 75% and the space group R3m; and a sixth compound having a ratio of the element X of 100% and the space group F4 3m. A size of the cells may be about 10 Å to about 50 Å.

In simulating the random structure, the plurality of compounds may be differentially distributed by locating any one compound in any one cell. In simulating the random structure, among the plurality of compounds, a compound having higher thermodynamic stability may be distributed to a larger number of the cells, so that a ratio of the compound occupying the random structure is increased.

In calculating the potential of the random structure, the potential of the random structure is calculated using van der Waals (vdW)-corrected semilocal xc functional (optB88).

The potential of the random structure may be a Moment Tensor Potential (MTP).

In one exemplary embodiment, the present disclosure provides a solid electrolyte having an argyrodite-type crystal structure and expressed as Lis-aPS5-aX1+a (0≤ a≤1, and X=Cl, Br, or I).

In another exemplary embodiment, the present disclosure provides a method of predicting lithium ion conductivity of a solid electrolyte, including simulating a plurality of compounds having different ratios of an element X occupying 4c sites included in an argyrodite-type crystal structure, assuming a virtual space including nxm×p cells (each of n, m and p being an integer in a range of 1 to 3), and simulating a random structure by differentially distributing the plurality of compounds to the cells based on thermodynamic stabilities of the respective compounds, calculating a potential specific to the random structure, and predicting the lithium ion conductivity of the solid electrolyte from the potential using molecular dynamics simulations.

In a preferred embodiment, the compounds may include a first compound having a ratio of the element X of 0% and a space group F43m, a second compound having a ratio of the element X of 25% and a space group R3m, a third compound having a ratio of the element X of 50% and a space group P2₁22, a fourth compound having a ratio of the element X of 50% and a space group P2 mm, a fifth compound having a ratio of the element X of 75% and the space group R3m, and a sixth compound having a ratio of the element X of 100% and the space group F43m.

In another preferred embodiment, a size of the cells may be about 10 Å to about 50 Å.

In still another preferred embodiment, in simulating the random structure, the plurality of compounds may be differentially distributed by locating any one compound in any one cell.

In yet another preferred embodiment, in simulating the random structure, among the plurality of compounds, a compound having higher thermodynamic stability may be distributed to a larger number of the cells, so that a ratio of the compound occupying the random structure is increased.

In still yet another preferred embodiment, in calculating the potential of the random structure, the potential of the random structure may be calculated using van der Waals (vdW)-corrected semilocal xc functional (optB88).

In a further preferred embodiment, the potential of the random structure may be a Moment Tensor Potential (MTP).

In another further preferred embodiment, in predicting the lithium ion conductivity, the lithium ion conductivity of the solid electrolyte at a temperature of about 300 K or higher may be predicted.

In still another further preferred embodiment, in predicting the lithium ion conductivity, the lithium ion conductivity of the solid electrolyte having a degree of crystallinity (Xc) of 0.7 to 0.8 may be predicted.

Other embodiments and preferred embodiments of the invention are discussed infra.

The above and other features of the invention are discussed infra.

### BRIEF DESCRIPTION OF THE DRAWINGS

The above and other features of the present disclosure will now be described in detail with reference to certain exemplary embodiments thereof illustrated in the accompanying drawings which are given hereinbelow by way of illustration only, and thus are not limitative of the present disclosure, and wherein:

FIG. 1 shows an argyrodite-type crystal structure of a solid electrolyte according to the present disclosure;

FIG. 2 shows crystal structures of a plurality of compounds according to the present disclosure;

FIG. 3 shows a random structure according to the present disclosure; and

FIG. 4 shows results of measurement of ion diffusivity during a process of predicting lithium ion conductivity of a solid electrolyte according to the present disclosure.

It should be understood that the appended drawings are not necessarily to scale, presenting a somewhat simplified representation of various preferred features illustrative of the basic principles of the invention. The specific design features of the present disclosure as disclosed herein, including, for example, specific dimensions, orientations, locations, and shapes, will be determined in part by the particular intended application and use environment.

In the figures, reference numbers refer to the same or equivalent parts of the present disclosure throughout the several figures of the drawing.

### DETAILED DESCRIPTION

The above-described objects, other objects, advantages and features of the present disclosure will become apparent from the descriptions of embodiments given hereinbelow with reference to the accompanying drawings. However, the present disclosure is not limited to the embodiments disclosed herein and may be implemented in various different forms. The embodiments are provided to make the description of the present disclosure thorough and to fully convey the scope of the present disclosure to those skilled in the art.

In the following description of the embodiments, the same elements are denoted by the same reference numerals even when they are depicted in different drawings. In the drawings, the dimensions of structures may be exaggerated compared to the actual dimensions thereof, for clarity of description. In the following description of the embodiments, terms, such as “first” and “second”, may be used to describe various elements but do not limit the elements. These terms are used only to distinguish one element from other elements. For example, a first element may be named a second element, and similarly, a second element may be named a first element, without departing from the scope and spirit of the invention. Singular expressions may encompass plural expressions, unless they have clearly different contextual meanings.

In the following description of the embodiments, terms, such as “including”, “comprising” and “having”, are to be interpreted as indicating the presence of characteristics, numbers, steps, operations, elements or parts stated in the description or combinations thereof, and do not exclude the presence of one or more other characteristics, numbers, steps, operations, elements, parts or combinations thereof, or possibility of adding the same. In addition, it will be understood that, when a part, such as a layer, a film, a region or a plate, is said to be “on” another part, the part may be located “directly on” the other part or other parts may be interposed between the two parts. In the same manner, it will be understood that, when a part, such as a layer, a film, a region or a plate, is said to be “under” another part, the part may be located “directly under” the other part or other parts may be interposed between the two parts.

All numbers, values and/or expressions representing amounts of components, reaction conditions, polymer compositions and blends used in the description are approximations in which various uncertainties in measurement generated when these values are acquired from essentially different things are reflected and thus it will be understood that they are modified by the term “about”, unless stated otherwise. Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”. In addition, it will be understood that, if a numerical range is disclosed in the description, such a range includes all continuous values from a minimum value to a maximum value of the range, unless stated otherwise. Further, if such a range refers to integers, the range includes all integers from a minimum integer to a maximum integer, unless stated otherwise.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit”, “-er”, “-or”, and “module” described in the specification mean units for processing at least one function and operation, and can be implemented by hardware components or software components and combinations thereof.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer readable media on a computer readable medium containing executable program instructions executed by a processor, controller or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network coupled computer systems so that the computer readable media is stored and executed in a distributed fashion, e.g., by a telematics server or a Controller Area Network (CAN).

A method of predicting lithium ion conductivity of a solid electrolyte according to the present disclosure may be a method of predicting the lithium ion conductivity of the solid electrolyte which has an argyrodite-type crystal structure and is expressed as Li6-aPS5-aX1+a (0≤a≤1, and X=Cl, Br, or I).

The above method may include simulating a plurality of compounds having different ratios of an element X occupying 4c sites included in the argyrodite-type crystal structure (S10), assuming a virtual space including n×m×p cells (each of n, m and p being an integer in the range of 1 to 3) and simulating a random structure by differentially distributing the respective compounds to the respective cells based on thermodynamic stabilities of the respective compounds (S20), calculating a potential of the random structure (S30), and predicting the lithium ion conductivity of the solid electrolyte from the potential using the molecular dynamics simulations.

Hereinafter, the respective operations will be described in detail

### Production of Compounds (S10)

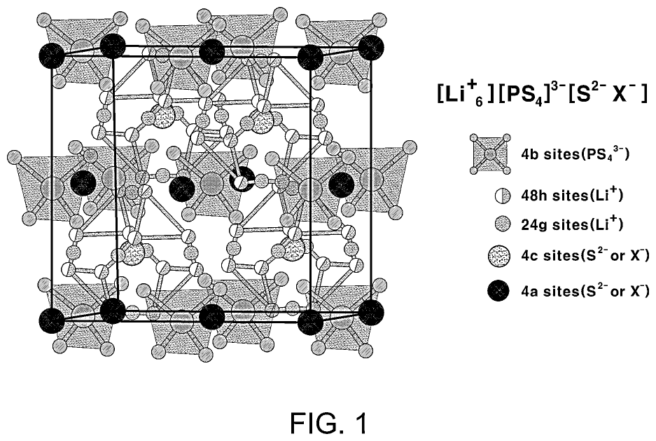

FIG. 1 shows the argyrodite-type crystal structure of the solid electrolyte according to the present disclosure. The argyrodite-type crystal structure may include 4b sites, 48h sites, 24g sites, 4c sites, and 4a sites. The solid electrolyte according to the present disclosure expressed as Li₆₋ₐPS₅₋ₐX₁₊ₐ (0≤ a≤1, and X=Cl, Br, or I) may be expressed as [Li⁺₆] [PS₄]³⁻[S²⁻X⁻].

PS₄³⁻ having a polyhedral shape may be located at the 4b sites. Lit may occupy all the 24g sites and some of the 48h sites, and may thus form the shape of a kind of cage.

Each of anions S²⁻ and X may be located at the 4a sites or the 4c sites, thereby being capable of forming a shape in which these anions are confined in the cage formed by lithium. The lithium ion conductivity of the solid electrolyte may be varied depending on how S²⁻ and X are located at the 4a sites and the 4c sites.

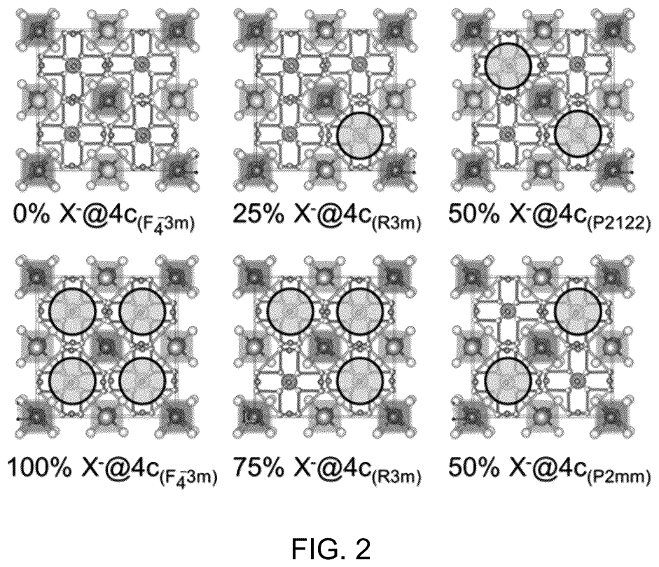

First, a basic lattice structure may be obtained by forming lithium sites using an enumlib code (http://github.com/msg-byu/enumlib). Thereafter, compounds having ratios of the element X occupying the 4c sites, which are 0%, 25%, 50%, 75% and 100%, may be designed. FIG. 2 shows crystal structures of the plurality of compounds according to the present disclosure. Concretely, the compounds may include a first compound having a ratio of the element X of 0% and a space group F43m, a second compound having a ratio of the element X of 25% and a space group R3m, a third compound having a ratio of the element X of 50% and a space group P2122, a fourth compound having a ratio of the element X of 50% and a space group P2 mm, a fifth compound having a ratio of the element X of 75% and the space group R3m, and a sixth compound having a ratio of the element X of 100% and the space group F43m.

### Production of Random Structure (S20)

When the plurality of compounds is produced as above, in a conventional method of predicting lithium ion conductivity of a solid electrolyte, lithium ion conductivities of the plurality of compounds are calculated and then lithium ion conductivity of the solid electrolyte in a bulk state is predicted based on calculation results, and thus, the conventional method is very expandable, requires a long time, and is not accurate.

The present disclosure has been made in an effort to solve the above-described problems of the conventional method, and is characterized in that the random structure including the plurality of compounds in a specific ratio is produced, the potential of the random structure is calculated, and the lithium ion conductivity of the solid electrolyte is predicted based on the calculated potential.

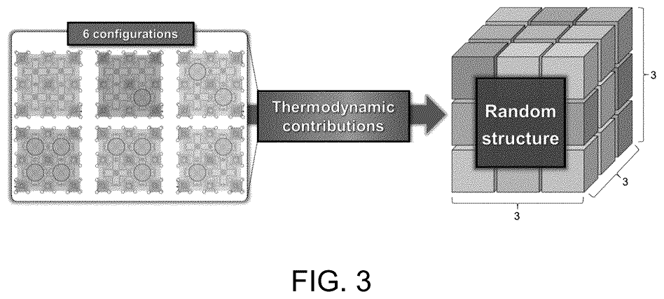

FIG. 3 shows the random structure according to the present disclosure.

First, a supercell structure including nxm×p cells (each of n, m and p being an integer in the range of 1 to 3) may be modeled. FIG. 3 exemplarily shows a supercell structure including 3×3×3 arrangement.

The size of the cells is not limited to a specific size, and may be, for example, about 10 Å to about 50 Å.

The random structure may simulate an actual solid electrolyte in a bulk state. A compound having the most thermodynamically stable crystal structure may occupy the largest part of the configuration of the solid electrolyte in the bulk state. That is, the actual solid electrolyte in the bulk state may be simulated as the random structure by differentially distributing the plurality of compounds to the cells based on the thermodynamic stabilities of the respective compounds. Here, only one compound may be assigned to one cell.

How many cells a specific compound is assigned to may be calculated by the following Equation.

$$Number of site = {P}_{i} ( E ) * {n}_{s} / sum of {P}_{i} ( E )$$

Here, nₛ may be the number of the cells forming the random structure. When the random structure shown in FIG. 3 is modeled, nₛ may be 27.

Pᵢ(E) may indicate thermodynamic contribution, and may be calculated by the following Equation.

$${P}_{i} ( E ) ∝ {e}^{\frac{- Δ E}{kT}}$$

Here, ΔE may indicate relative thermodynamic stability of each component to the most stable component using density functional theory (DFT). k may indicate the Boltzmann constant. T may indicate temperature.

Consequently, in simulating the random structure, among the plurality of compounds, a compound having higher thermodynamic stability may be distributed to a larger number of cells, and thus the ratio of the compound occupying the random structure may be increased.

### Calculation of Potential of Random Structure (S30)

In order to calculate the lithium ion conductivity of the solid electrolyte using the molecular dynamics simulations, the potential of the random structure may be required. The potential may mean force between atoms forming the corresponding solid electrolyte. The potential of the random structure may be a Moment Tensor Potential (MTP).

As functionals to calculate the MTP, there are the Perdew-Burke-Ernzerhof (PBE) functional (referred to hereinafter as “PBE), the PBE functional with DFT-D3 (referred to hereinafter as “DFT-D3”), van der Waals (vdW)-corrected semilocal xc functional (referred to hereinafter as “optB88”), etc. The present disclosure is characterized in that the potential of the random structure is calculated using optB88.

Recently, machine learning potentials are being researched as a method to replace density functional theory (DFT) which is comparatively accurate but has high computational cost. Among many machine learning potentials, such as a Neural Network Potential (NNP), a Gaussian Approximation Potential (GAP), a Spectral Neighbor Analysis Potential (SNAP), and the like, the present disclosure is characterized in that a Moment Tensor Potential (MTP) is used.

The Moment Tensor Potential (MTP) may mean potential energy of a crystal structure in the machine learning potential. The potential energy may mean the sum total of energies, which the respective atoms will have, in consideration of local atomic environments of the respective atoms by the following Equation.

$${E}^{mtp} = {\sum }_{i}^{n} V ( {n}_{i} )$$

Here, nᵢ may indicate the local atomic environment of an i^{th} atom. The local atomic environment may indicate the atomic type and position of the i^{th} atom, and the atomic type and position of a j^{th} atom around the i^{th} atom.

V (nᵢ) may be expressed as the following Equation.

$$V ( {n}_{i} ) = {\sum }_{α} {ξ}_{α} {B}_{α} ( {n}_{i} )$$

Here, ξₐ may be a constant which fits a training process in the Machine-Learning Interatomic Potential (MLIP) package. α may the sum total of basis functions of the respective atoms. Ba may be defined as the following Equation.

$${levM}_{μ , v} = 2 + 4 μ + v$$

Here, u and v may be coefficient terms in the Machine-Learning Interatomic Potential (MLIP) package. As the values of μ and v increase, calculation accuracy may increase, but the computational cost may be considerably increased. M_{μ,v} may be a moment tensor descriptor, and may be expressed as the following Equation.

$${M}_{μ , v} ( {n}_{i} ) = {\sum }_{j} {f}_{μ} ( \vert {r}_{i j} \vert , {z}_{i} , {z}_{j} ) {r}_{ij} ⊗ … ⊗ {r}_{ij}$$

Here, zᵢ and zⱼ may indicate the atomic types of the i^{th} and j^{th} atoms, and rᵢⱼ may indicate the vector of the j^{th} atom to the i^{th} atom. The above Equation may be divided into a radial part expressed as f_{μ}, and an angular part expressed as rᵢⱼ ⊗ . . . ⊗rᵢⱼ. In the radial part, results depending on an interatomic distance, which is a cutoff radius R_{cut} to designate a distance including a neighboring atom based on the i^{th} atom, and the atomic types may be obtained.

In the present disclosure, hyperparameters for machine learning are set as below. Concretely, the Moment Tensor Potential (MTP) of the random structure may be obtained by performing potential fitting by setting the cutoff radius R_{cut} to 5 Å, levₘₐₓ to 8, the weight ratio of energy to force to 100:1, and the ratio of a training set to a validation set to 9:1. Prediction of Lithium Ion conductivity (S40)

The molecular dynamics simulations may be performed using the Moment Tensor Potential (MTP) produced in the former operation. The molecular dynamics simulations may be performed using a Large-scale Atomic/Molecular Massively Parallel Simulator (LAMMPS) package.

According to the present disclosure, the lithium ion conductivity of the solid electrolyte at room temperature of about 300 K may be predicted. Since the lithium ion conductivity of the solid electrolyte is low at room temperature, in the conventional method, the lithium ion conductivity of the solid electrolyte at a high temperature of about 700 K was predicted, but the present disclosure is characterized in that the lithium ion conductivity of the solid electrolyte at room temperature of about 300 K is predicted by extrapolation from the lithium ion conductivities of the solid electrolyte at high temperatures using Arrhenius fitting. Concretely, in the present disclosure, the lithium ion conductivities of the solid electrolyte at temperatures of about 300 K to about 1,800 K may be predicted.

In order to increase accuracy in prediction, the molecular dynamics simulations at seven temperatures, i.e., temperatures of about 350 K to about 500 K at intervals of 25 K, may be performed. In order to find the averages, two calculations may be repeatedly performed under the same conditions.

Diffusivity of lithium ions is low at a temperature of about 350 K, about 375 K, or the like, and thus migration of the lithium ions may be insufficient at this temperature. The rate of change of the mean square displacement (MSD) of lithium ions over time is very low at a low temperature. Therefore, temperatures at which the rate of change of the mean square displacement (MSD) of lithium ions over time is low are excluded from calculation of the lithium ion conductivity, and additional temperatures starting from about 500 K at intervals of about 25 K may be included in order to obtain diffusivity at higher temperatures. In the molecular dynamics simulations, NVT simulations may be performed with a time step of 1 fs from about 10 K to a target temperature in order to achieve system balance for 100 ps. Thereafter, NPT simulations may be performed at the target temperature for 10 nₛ or more. Diffusivities may be obtained from the MSDs through two repeated NPT simulations, and the lithium ion conductivity of the solid electrolyte at about 300 K may be obtained by fitting of the diffusivities obtained at the respective temperatures using the Arrhenius equation. Hereinafter, a method of calculating the lithium ion conductivity will be described in detail.

In order to obtain diffusivity of lithium ions, the mean square displacement (MSD) representing deviation of particle positions based on a reference position over time should be calculated. The diffusivity of the lithium ions may be calculated from the mean square displacement (MSD) through the following equation in calculations using the Large-scale Atomic/Molecular Massively Parallel Simulator (LAMMPS) package.

$$D = \frac{1}{2 dt} MSD$$

Here, D may be diffusivity of lithium ions. Since diffusivity of ions in a solid having no phase transition follows the following Arrhenius equation, the diffusivity of lithium ions at a specific temperature may be calculated. That is, the diffusivity of lithium ions at about 300 K may be inferred through Arrhenius fitting using diffusivities obtained by simulations at high temperatures (generally about 600 K to 1,200 K).

$$D = {D}_{0} exp ( - \frac{{E}_{a}}{kT} )$$

Here, Do may indicate diffusivity of lithium ions at a starting temperature. Eₐ may be activation energy of diffusion. K may be the Boltzmann constant. T may indicate temperature. Eₐ may be calculated by linear regression of a graph plotted between log D and 1/T.

The lithium ion conductivity of the solid electrolyte at about 300 K may be finally calculated by substituting the above-calculated diffusivity at about 300 K into the following Einstein diffusion equation.

$${σ}_{T} = \frac{ρ {z}^{2} {F}^{2}}{RT} {D}_{T}$$

Here, p may be the density of diffused ions in a unit cell, z may be the charge of the diffused ions, F may be the Faraday constant, and R may be the gas constant.

Hereinafter, the present disclosure will be described in more detail through the following test examples. The following test examples serve merely to exemplarily describe the present disclosure, and are not intended to limit the scope of the invention.

Hereinafter, comparative analysis results of accuracies in PBE, DFT-D3 and optB88 will be described.

First, 6 compounds were produced in the same manner as Operation S10. In order to make training sets covering a variety of cases, a total of 18 training sets was produced by additionally modeling structures obtained by applying strain of +5% to each compound in all lattice directions. Further, temperatures at which diffusivity is calculated were set to about 300 K, 600 K, 900 K, and 1,200 K, and a total of 7,200 training sets was completed by picking 100 structure at each of the temperatures.

A solid electrolyte having the argyrodite-type crystal structure and expressed as Li6PS₅X (X being Cl, Br, or I) is structurally varied depending on the ratio of a halogen element occupying the 4a sites and 4c sites. The solid electrolyte may exhibit very low lithium ion conductivity in ordered structures in which the ratio of the halogen element occupying the 4c sites is 0% and 100%, and may exhibit very high lithium ion conductivity in disordered structures in which the ratio of the halogen element occupying the 4c sites is 25%, 50%, and 75%. When potentials calculated by the functionals, such as PBE, DFT-D3 and optB88, are reasonable, these potentials should be able to obviously represent such a difference.

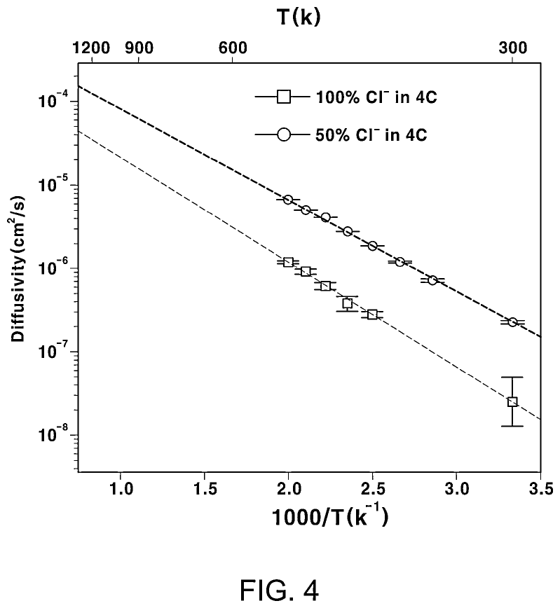

First, training sets in which the ratio of the halogen element occupying the 4c sites is 100% and 50% were produced, and lithium ion conductivities ORT of structures of Li₆PS₅Cl at room temperature of about 300 K were calculated based on Moment Tensor Potentials (MTP) obtained through PBE. Results are set forth in Table 1 below and FIG. 4.

TABLE 1

| | | Site occupancy | | σ_{RT} |
|---|---|---|---|---|
| Composition | Functional | (X⁻ @, 4 c site) | | [mS/cm] |
| Li₆PS₅Cl | MTP-PBE | 100% | (F4̅3m) | 4 |
| | | 50% | (P2₁22) | 34 |
| | DFT-PBE | 100% | (F4̅3m) | 0.57 |
| | | 50% | (P2₁22) | 115 |

Referring to Table 1 and FIG. 4, a tendency of the lithium ion conductivities calculated based on MTP-PBE coincides with a tendency of lithium ion conductivities calculated based on DFT-PBE but, in the structure in which the ratio of the halogen element occupying the 4c sites is 100%, the lithium ion conductivity calculated based on MTP-PBE differs greatly from an experimental value, i.e., 1 mS/cm, and is higher than the lithium ion conductivity calculated based on DFT-PBE, and therefore, it may be confirmed that Moment Tensor Potential (MTP) calculation through PBE is not suitable for prediction of the lithium ion conductivity of the solid electrolyte having the argyrodite-type crystal structure.

Thereafter, potentials were calculated through optB88 and DFT-D3 using the above-produced 7,200 training sets, and lithium ion conductivities ORT at room temperature (about 300 K) were predicted from the potentials, and were compared to values calculated by the AIMD method. Results of prediction are set forth in Table 2 below.

TABLE 2

| | Site occupancy | | Eᵣₑₗ | Eₐ | σ_{RT} |
|---|---|---|---|---|---|
| Functional | (X⁻ @, 4 c site) | | [meV/atom] | [meV] | [mS/cm] |
| DFT | 0% | (F4̅3m) | 23 | 452 | 0.02 |
| AIMD | 25% | (R3m) | 4 | 193 | 45 |
| | 50% | (P2₁22) | 0 | 160 | 115 |
| | 50% | (P2mm) | 11 | 151 | 183 |
| | 75% | (R3m) | 13 | 184 | 60 |
| | 100% | (F4̅3m) | 206 | 339 | 0.57 |
| | σ_{bulk}: 18.50 mS/cm, σ_{80%}: 3.76 mS/cm | | | | |
| MTP | 0% | (F4̅3m) | 23 | 502 | 0.008 |
| optB88 | 25% | (R3m) | 4 | 220 | 19.2 |
| | 50% | (P2₁22) | 0 | 256 | 12.7 |
| | 50% | (P2mm) | 11 | 246 | 15.7 |
| | 75% | (R3m) | 13 | 250 | 13.3 |
| | 100% | (F4̅3m) | 206 | 406 | 0.01 |
| | σ_{bulk}: 12.10 mS/cm, σ_{80%}: 2.46 mS/cm | | | | |
| MTP | 0% | (F4̅3m) | 23 | 997 | Very low |
| DFT-D3 | 25% | (R3m) | 4 | 286 | 3.6 |
| | 50% | (P2₁22) | 0 | 290 | 2.7 |
| | 50% | (P2mm) | 11 | 290 | 4.4 |
| | 75% | (R3m) | 13 | 337 | 1.9 |
| | 100% | (F4̅3m) | 206 | 271 | 3.7 |

| | |
|---|---|
| | σ_{bulk}: 2.72 mS/cm, σ_{80%}: 0.55 mS/cm |
| — | σ_{Expt}: 2.2 mS/cm |

In Table 2, Eᵣₑₗ represents relative energy of each structure to the most stable structure by comparing DFT optimization energies, and were expressed as the same values in the AIMD and MTP methods. σ_{bulk} is a value obtained by statistically reflecting thermodynamic contributions and kinetic contributions of six compounds having different ratios of the halogen element occupying the 4c sites, in order to express the lithium ion conductivity of the solid electrolyte in the bulk state using the lithium ion conductivities of the respective compounds. σ_{80%} is the lithium ion conductivity of the solid electrolyte on the assumption that the degree of crystallinity X_{c} of the solid electrolyte is 0.8 (80%). In general, the lithium ion conductivity of the solid electrolyte on the assumption that the degree of crystallinity of the solid electrolyte predicted through simulations is 80% may almost coincide with actual lithium ion conductivity of the solid electrolyte. In the present disclosure, the lithium ion conductivity of the solid electrolyte having a degree of crystallinity X_{c} of 0.7 to 0.8 may be predicted.

Referring to Table 2, it may be confirmed that the lithium ion conductivities ØRT of ordered structures in which the ratio of the halogen element occupying the 4c sites was 0% and 100%, calculated based on optB88 were very low, i.e., 0.008 and 0.01 mS/cm, and it was predicted that the ordered structures have low lithium ion conductivities, as in results of the conventional DFT-AIMD method. Further, the lithium ion conductivity 080% of the solid electrolyte predicted based on optB88 was 2.46 mS/cm, which is almost the same as a reported experimental value. Therefore, it may be confirmed that Moment Tensor Potential (MTP) calculation and lithium ion conductivity prediction using optB88 are very reasonable.

On the other hand, referring to results using DFT-D3, the calculated lithium ion conductivity of an ordered structure in which the ratio of the halogen element occupying the 4c sites was 0% was very low, but the calculated lithium ion conductivity of an ordered structure in which the ratio of the halogen element occupying the 4c sites was 100% was high, i.e., 3.7 mS/cm. Therefore, it may be confirmed that DFT-D3 is not suitable for prediction of the lithium ion conductivity of the solid electrolyte according to the present disclosure.

Lithium ion conductivity of Li₆PS₅Br was predicted using optB88 by the same method as above. Results of prediction are set forth in Table 3 below.

TABLE 3

| | Site occupancy | | Eᵣₑₗ | Eₐ | σ_{RT} |
|---|---|---|---|---|---|
| Functional | (X⁻ @, 4 c site) | | [meV/atom] | [meV] | [mS/cm] |
| DFT | 0% | (F4̅3m) | 23 | 557 | 0.0005 |
| AIMD | 25% | (R3m) | 0 | 219 | 16 |
| | 50% | (P2₁22) | 11 | 194 | 42 |
| | 50% | (P2mm) | 39 | 196 | 51 |
| | 75% | (R3m) | 33 | 188 | 51 |
| | 100% | (F4̅3m) | 251 | 401 | 0.08 |
| | σ_{bulk}: 15.26 mS/cm, σ_{80%}: 3.10 mS/cm | | | | |
| MTP | 0% | (F4̅3m) | 23 | 467 | 0.01 |
| optB88 | 25% | (R3m) | 0 | 219 | 12.9 |
| | 50% | (P2₁22) | 11 | 288 | 7.7 |
| | 50% | (P2mm) | 39 | 256 | 12.4 |
| | 75% | (R3m) | 33 | 300 | 5.7 |
| | 100% | (F4̅3m) | 251 | 447 | 0.05 |

σ_{bulk}: 7.81 mS/cm, σ_{80%}: 1.59 mS/cm

Similarly to Li₆PS₅Cl, in case of Li₆PS₅Br, the calculated lithium ion conductivities ORT of ordered structures in which the ratio of the halogen element occupying the 4c sites was 0% and 100%, were low. Further, the lithium ion conductivity 080% of the solid electrolyte predicted based on optB88 was 1.59 mS/cm, which is very similar to an experimental value. Therefore, it may be confirmed that the method using optB88 is also very reasonable in Li₆PS₅Br.

Further, lithium ion conductivity of Li₆PS₅I was predicted using optB88 by the same method. Results of prediction are set forth in Table 4 below.

TABLE 4

| | Site occupancy | | Eᵣₑₗ | Eₐ | σ_{RT} |
|---|---|---|---|---|---|
| Functional | (X⁻ @ 4 c site) | | [meV/atom] | [meV] | [mS/cm] |
| DFT | 0% | (F4̅3m) | 0 | 695 | 3 × 10⁻⁶ |
| AIMD | 25% | (R3m) | 22 | 255 | 4.8 |
| | 50% | (P2₁22) | 37 | 202 | 30 |
| | 50% | (P2mm) | 47 | 227 | 17 |
| | 75% | (R3m) | 49 | 221 | 17 |
| | 100% | (F4̅3m) | 284 | 531 | 0.001 |
| | σ_{bulk}: 4.17 mS/cm, σ_{80%}: 0.85 mS/cm | | | | |
| MTP | 0% | (F4̅3m) | 0 | 695 | 1 × 10⁻⁶ |
| optB88 | 25% | (R3m) | 22 | 255 | 8.3 |
| | 50% | (P2₁22) | 37 | 202 | 15.6 |
| | 50% | (P2mm) | 47 | 227 | 17.8 |
| | 75% | (R3m) | 49 | 221 | 7.1 |
| | 100% | (F4̅3m) | 284 | 531 | 0.001 |

σ_{bulk}: 4.17 mS/cm, σ_{80%}: 0.85 mS/cm

It is known that Li₆PS₅I has a very low lithium ion conductivity, and the reason for this is that a structure in which the ratio of the halogen element occupying the 4c sites is 0% is thermodynamically stable and thus six structures are not mixed but only the structure having the ratio of the halogen element of 0% is present in a bulk structure. Therefore, the predicted result of the lithium ion conductivity of the structure having the ratio of the halogen element of 0% coincides with an experimental value, and thus, it may be confirmed that the method using optB88 is also reasonable in Li₆PS₅I.

Thereafter, random structures were produced using a plurality of structures, potentials of the random structures were calculated based on optB88, and lithium ion conductivities of solid electrolytes were predicted using the molecular dynamics simulations.

In the solid electrolytes expressed as Li₆PS₅CI, Li₆PS₅Br, and Li₆PS₅I, the above-described six compounds were produced by varying the ratio of a halogen element occupying 4c sites. A supercell including 3×3×3 cells shown in FIG. 3 was modeled, and the numbers of cells occupied by the respective compounds of each solid electrolyte were calculated based on relative structural stabilities of the respective compounds, as set forth in Table 5 below.

TABLE 5

| | Site occupancy | | Eᵣₑₗ | | Number of |
|---|---|---|---|---|---|
| Composition | (X⁻ @ 4 c site) | | [meV/atom] | Pᵢ(E) | contributions |
| Li₆PS₅Cl | 0% | (F4̅3m) | 23 | 0.41 | 3 |
| | 25% | (R3m) | 0 | 0.88 | 7 |
| | 50% | (P2₁22) | 11 | 1.00 | 7 |
| | 50% | (P2mm) | 39 | 0.65 | 5 |
| | 75% | (R3m) | 33 | 0.61 | 5 |
| | 100% | (F4̅3m) | 251 | 0.00 | 0 |
| Li₆PS₅Br | 0% | (F4̅3m) | 23 | 0.41 | 4 |
| | 25% | (R3m) | 0 | 1.00 | 11 |
| | 50% | (P2₁22) | 11 | 0.65 | 7 |
| | 50% | (P2mm) | 39 | 0.22 | 2 |
| | 75% | (R3m) | 33 | 0.28 | 3 |
| | 100% | (F4̅3m) | 251 | 0.00 | 0 |
| Li₆PS₅I | 0% | (F4̅3m) | 23 | 1.00 | 14 |
| | 25% | (R3m) | 0 | 0.42 | 6 |
| | 50% | (P2₁22) | 11 | 0.24 | 3 |
| | 50% | (P2mm) | 39 | 0.16 | 2 |
| | 75% | (R3m) | 33 | 0.15 | 2 |
| | 100% | (F4̅3m) | 251 | 0.00 | 0 |

Referring to Table 5 above, in Li₆PS₅Cl, the compound having a ratio of the halogen element of 25% (R3m) and the compound having a ratio of the halogen element of 50% (P2122), which have high thermodynamic contributions Pᵢ (E), occupied 7 cells among total 27 cells, respectively, and next, the compound having a ratio of the halogen element of 50% (P2 mm) and the compound having a ratio of the halogen element of 75% (R3m) occupied 5 cells, respectively.

The random structures were produced by assigning the respective compounds to the cells.

Moment Tensor Potentials (MTP) of the random structures were calculated based on optB88, and the lithium ion conductivities thereof were calculated by performing the molecular dynamics simulations using the Moment Tensor Potentials (MTP). Results of calculation are set forth in Table 6 below.

TABLE 6

| | | σ_{RT} | σ_{80%} | σₑₓₚ |
|---|---|---|---|---|
| Composition | Functional | [mS/cm] | [mS/cm] | [mS/cm] |
| Li₆PS₅Cl | optB88 | 11.5 | 2.3 | 2.17 |
| Li₆PS₅Br | | 8.8 | 1.8 | 0.99 |
| Li₆PS₅I | | 2.6 | 0.5 | 0.00223 |
| Li₆PS₅Cl | DFT-D3 | 3.1 | 0.6 | 2.17 |

Referring to Table 6, it may be confirmed that the lithium ion conductivities 080% of Li₆PS₅Cl and Li₆PS₅Br are very similar to experimental values σₑₓₚ. The lithium ion conductivity 080% of Li₆PS₅l is somewhat higher than an experimental value σₑₓₚ. It is determined that the reason for such a difference is that a structure in which the ratio of the halogen element occupying the 4c sites is 0% is thermodynamically stable and thus the six structures are not actually mixed but only the structure having the ratio of the halogen element of 0% is present in an actual structure.

The lithium ion conductivity 080% of the random structure calculated based on DFT-D3 was 0.62 mS/cm which considerably differs from the experimental value. That is, as described above, it may be confirmed that DFT-D3 is not suitable for prediction of the lithium ion conductivity of the solid electrolyte having the argyrodite-type crystal structure.

As is apparent from the above description, according to the present disclosure, lithium ion conductivity of a solid electrolyte may be rapidly and precisely calculated.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

What is claimed is:

1. A method of predicting lithium ion conductivity of a solid electrolyte, the method comprising:
simulating a crystal structure of the solid electrolyte and creating training sets based on the crystal structure for machine learning;
calculating a potential specific to the simulated crystal structure by machine learning using the training sets; and
predicting the lithium ion conductivity of the solid electrolyte from the potential using molecular dynamics simulations.

2. The method of claim 1, wherein the training sets are created using ab initio molecular dynamics (AIMD) method.

3. The method of claim 1, wherein the potential specific to the simulated crystal structure is a Moment Tensor Potential (MTP).

4. The method of claim 1, wherein the MTP is used to calculate lithium ion diffusivity, which is then used to predict the lithium ion conductivity.

5. A method of predicting lithium ion conductivity of a solid electrolyte having an argyrodite-type crystal structure and expressed as Li₆₋ₐPS₅₋ₐX₁₊ₐ (0≤ a≤1, and X=Cl, Br, or I), comprising:
simulating a plurality of compounds having different ratios of an element X occupying 4c sites comprised in the argyrodite-type crystal structure;
assuming a virtual space comprising n×m×p cells (each of n, m and p being an integer in a range of 1 to 3), and simulating a random structure by differentially distributing the plurality of compounds to the cells based on thermodynamic stabilities of the plurality of compounds;
creating training sets based on the random structure for machine learning;
calculating a potential specific to the random structure by machine learning using the training sets; and
predicting the lithium ion conductivity of the solid electrolyte from the potential using molecular dynamics simulations.

6. The method of claim 5, wherein the training sets are created using ab initio molecular dynamics (AIMD) method.

7. The method of claim 5, wherein the compounds comprise:
a first compound having a ratio of the element X of 0% and a space group F43m;
a second compound having a ratio of the element X of 25% and a space group R3m;
a third compound having a ratio of the element X of 50% and a space group P2122;
a fourth compound having a ratio of the element X of 50% and a space group P2 mm;
a fifth compound having a ratio of the element X of 75% and the space group R3m; and
a sixth compound having a ratio of the element X of 100% and the space group F43m.

8. The method of claim 5, wherein a size of the cells is about 10 Å to about 50 Å.

9. The method of claim 5, wherein, in simulating the random structure, the plurality of compounds is differentially distributed by locating any one compound in any one cell.

10. The method of claim 5, wherein, in simulating the random structure, among the plurality of compounds, a compound having higher thermodynamic stability is distributed to a larger number of the cells, so that a ratio of the compound occupying the random structure is increased.

11. The method of claim 5, wherein, in calculating the potential of the random structure, the potential of the random structure is calculated using van der Waals (vdW)-corrected semilocal xc functional (optB88).

12. The method of claim 5, wherein the potential of the random structure is a Moment Tensor Potential (MTP).

13. A method of predicting lithium ion conductivity of a solid electrolyte having an argyrodite-type crystal structure and expressed as Li₆₋ₐPS₅₋ₐX₁₊ₐ (0≤ a≤1, and X=Cl, Br, or I), comprising:
simulating a plurality of compounds having different ratios of an element X occupying 4c sites comprised in the argyrodite-type crystal structure;
assuming a virtual space comprising n×m×p cells (each of n, m and p being an integer in a range of 1 to 3), and simulating a random structure by differentially distributing the plurality of compounds to the cells based on thermodynamic stabilities of the plurality of compounds;
calculating a potential specific to the random structure; and
predicting the lithium ion conductivity of the solid electrolyte from the potential using molecular dynamics simulations.

14. The method of claim 13, wherein the compounds comprise:
a first compound having a ratio of the element X of 0% and a space group F43m;
a second compound having a ratio of the element X of 25% and a space group R3m;
a third compound having a ratio of the element X of 50% and a space group P2122;
a fourth compound having a ratio of the element X of 50% and a space group P2 mm;
a fifth compound having a ratio of the element X of 75% and the space group R3m; and
a sixth compound having a ratio of the element X of 100% and the space group F43m.

15. The method of claim 13, wherein a size of the cells is about 10 Å to about 50 Å.

16. The method of claim 13, wherein, in simulating the random structure, the plurality of compounds is differentially distributed by locating any one compound in any one cell.

17. The method of claim 13, wherein, in simulating the random structure, among the plurality of compounds, a compound having higher thermodynamic stability is distributed to a larger number of the cells, so that a ratio of the compound occupying the random structure is increased.

18. The method of claim 13, wherein, in calculating the potential of the random structure, the potential of the random structure is calculated using van der Waals (vdW)-corrected semilocal xc functional (optB88).

19. The method of claim 13, wherein the potential of the random structure is a Moment Tensor Potential (MTP).

20. The method of claim 13, wherein, in predicting the lithium ion conductivity, the lithium ion conductivity of the solid electrolyte at a temperature of about 300 K or higher and/or having a degree of crystallinity (xc) of 0.7 to 0.8 is predicted.
